(19) United States
(12) Patent Application Publication
(10) Pub. No.: US 2014/0012090 A1
(43) Pub. Date: Jan. 9, 2014

(54) ILLUMINATABLE MEDICAL RETAINING HOOK

(71) Applicants: Martina Heitland, Munich (DE); Blasius Gerg, Hohenthann (DE)

(72) Inventors: Martina Heitland, Munich (DE); Blasius Gerg, Hohenthann (DE)

(73) Assignees: Blasius Gerg, Hohenthann (DE); Martina Heitland, Munich (DE)

(21) Appl. No.: 13/823,664

(22) PCT Filed: Oct. 6, 2011
(86) PCT No.: PCT/EP11/04995

§ 371 Date: Sep. 18, 2013

(30) Foreign Application Priority Data
Oct. 7, 2010 (DE) 10 2010 047 835.0

(51) Int. Cl. A61B 19/00

(52) U.S. Cl. CPC A61B 19/5202
USPC 600/217

(57) ABSTRACT

A medical retaining hook for holding tissue open during surgical interventions. One embodiment includes an exchangeable hook element of a polycarbonate or polymethylmethacrylate material. A light source is attachable to the hook element.

### BACKGROUND

The invention relates to a medical retaining hook for holding tissue open during surgical interventions, including a hook element, which is attached to a handle and which is also identified as surgical retractor.

In response to surgical interventions, e.g., in the area of the abdomen or thorax, it is indispensably necessary to hold back, that is open, the surrounding tissue, which lies above a surgical site, for example. Retaining hooks, which consist substantially of an approximately right-angled hook element made of flat stainless steel material, which merges into a handle thickening on the holding-side end and which, on principle, is provided with a blunt tissue retaining lug on the free end, which faces the surgical site, are typically used for this purpose by the support personnel, for example an operating room nurse or an assistant physician.

A good illumination of the surgical site is crucial for the surgeon, who performs the surgery. The typical movable and adjustable overhead lamps on operating tables often do not fulfill the demands for a possibility of an in-situ lighting or illumination, respectively, of the surgical site, because two or three “observation heads” between the overhead lamp and the surgical site often cause a shadowing effect.

It is known to guide an optical fiber having a light emission surface on or in the vicinity of the free end of the tissue retaining hook along the upper side of the medical retaining hooks in question within a small metal tube. For the most part, this optical fiber is connected via a suitable coupling element and then further via a fiber optic cable to a cold light source, the power supply unit of which is located in the vicinity of the operating table and which, in turn, is fed via a power cable. This type of lighting of a surgical site via the retaining hook is unsatisfactory to the extent that one or mostly a plurality of additional cable connections are necessary for the in-situ illumination of the surgical site in addition to the plurality of tubes and cables on a surgical table, which are already present. Different embodiment alternatives of surgical retaining hooks (surgical retractors) are also known from publication U.S. Pat. No. 7,306,559 B2, in the case of which a metallic retaining blade, which can be fixed to a handle and which is bent approximately at a right angle, together with a light conductor strip, which is arranged thereabove in a layered manner, are fixed in a handle, in the housing of which a power source (battery, accumulator) can be inserted, which feeds light into the light conductor. In the area of the free retaining section of the retaining blade, the light conductor strip is designed as light emitter, e.g., in the form of a plurality of lamps, so that the tissue (blood vessel, tendon, bone, etc.), which is grabbed by the hook, can be identified, but so that a sufficient illumination of the immediate surgical site being ensured.

For these and other reasons there is a need for the present invention.

### BRIEF DESCRIPTION OF THE DRAWINGS

Exemplary embodiments of the invention, which correspond substantially to already tested prototypes, will be defined below by using the drawing.

FIG. 1 illustrates an illuminatable medical retaining hook for holding open tissue during surgical interventions according to a first embodiment of the invention.

FIG. 2 with

partial FIG. 2A illustrates the isometric view of a second embodiment of the illuminatable hook element of a medical retractor including features according to the invention;

partial FIG. 2B illustrates the top view onto the hook element according to partial FIG. 2A with sectional illustration illustrated therebelow, viewed in the direction of the arrows at section B-B in FIG. 2B;

partial FIG. 2C illustrates the side view of the hook element according to

FIG. 2A and

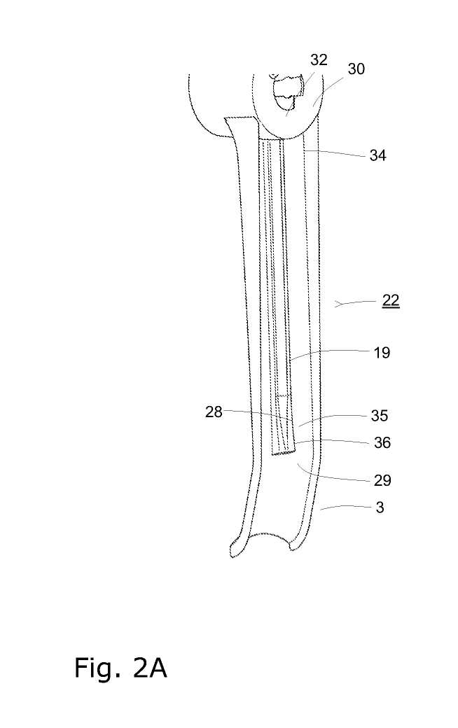

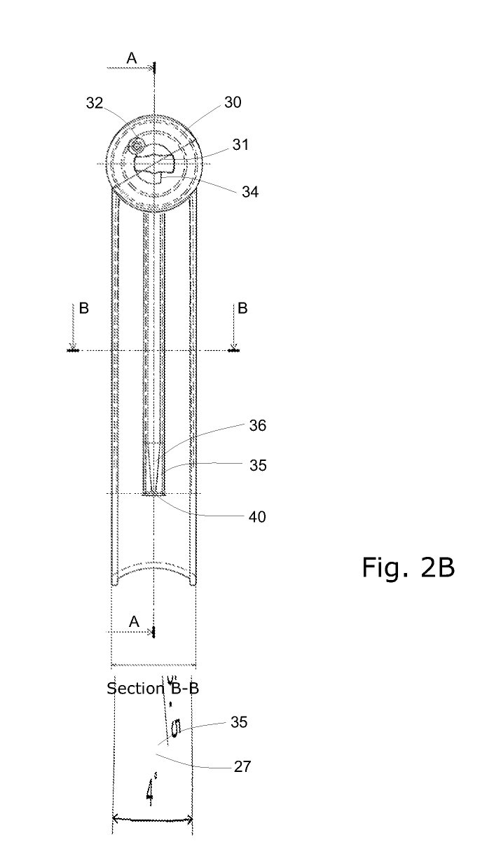

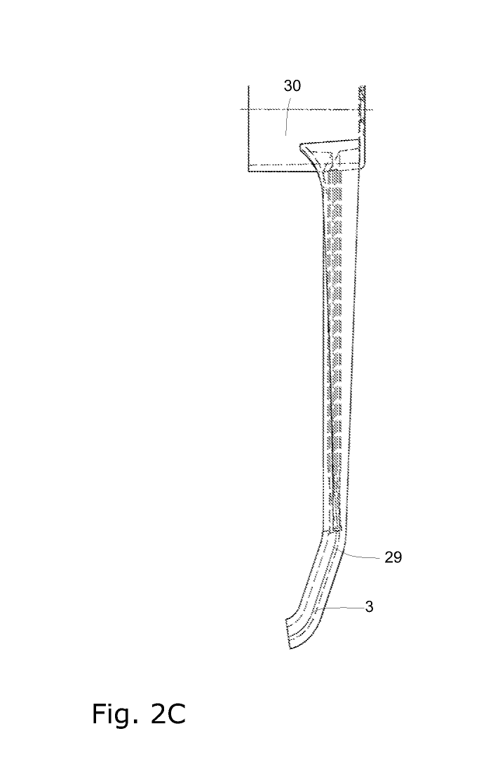

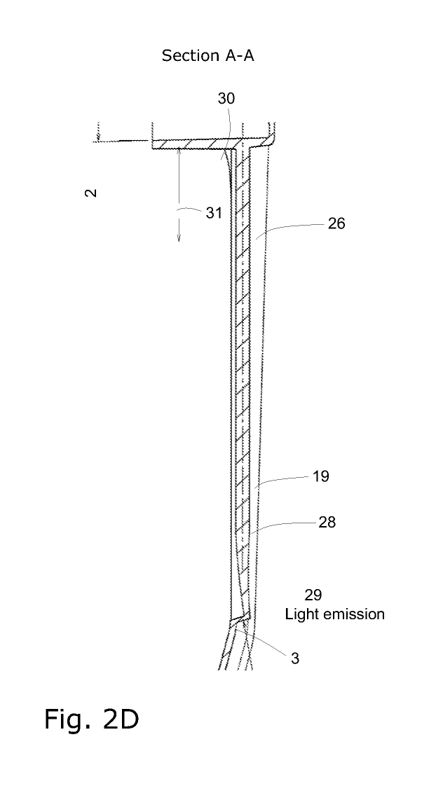

partial FIG. 2D illustrates the longitudinal sectional illustration of the hook element according to FIG. 2A, viewed in the direction of the arrows A-A onto the longitudinal section in FIG. 2B.

### DETAILED DESCRIPTION OF THE DRAWINGS

In the following Detailed Description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments can be positioned in a number of different orientations, the directional terminology is used for purposes of illustration and is in no way limiting. It is to be understood that other embodiments may be utilized and structural or logical changes may be made without departing from the scope of the present invention. The following detailed description, therefore, is not to be taken in a limiting sense, and the scope of the present invention is defined by the appended claims.

The present embodiments provide an autonomous medical hook element for holding tissue open, by which a good local illumination of the surgical sight can be ensured in response to a surgical intervention, even in response to a longer surgery time without additional heat emission.

In the case of a medical retaining hook for holding open tissue during surgical interventions by using a hook element, which can be attached to a handle, in the case of which the handle is designed as hollow body for accommodating an electrical energy storage means, and in the case of which a light source together with energy source and supply circuit, the emitted light of which is emitted into an irradiation surface on the front end side of a light conductor, which is guided along the hook element, and which is guided to the free end thereof, is inserted into the front end of the handle, which faces the hook element, the invention is characterized according to the invention in that the hook element, which at the same time serves as light conductor made of a light-conducting polycarbonate or polymethylmethacrylate material having a good heat resistance and optical clarity, high mechanical load-bearing capacity as well as impact toughness.

In one embodiment, the light source consists of a light-emitting diode (LED), which also emits white light or largely white light. An RGB laser (white light laser) can also be possible as light source.

It can be advantageous to insert a lens, which can in one embodiment be integrated into the flat irradiation end of the hook element, between the light emission surface of the light source, in one embodiment of the white light LED and the irradiation end of the hook element. To ensure a good spot light illumination of the surgical site, it can also be advantageous for provision to be made on the free light emission end or area for a lens, in one embodiment a converging lens, which can be adhered to or integrally molded in one piece to the end section of the light conductor, which in one embodiment already constricts the luminous flux.

The exterior design of the hook element itself does not fundamentally differ from the currently known hook elements, which have proven to work. It is produced, namely in one piece, including the connecting part, which is thickened on the handle side, for example including a crosswise slot on the inside for a bayonet connection with the handle, in a high-quality injection process, which ensures extremely smooth surfaces of a polycarbonate material or polymethylmethacrylate material (PMMA) having a good heat resistance and optical clarity. The hook element encompasses curved edges, which in one embodiment run in the longitudinal direction thereof, and possibly a beveled light reflection surface in the area of the main curvature of the hook element. On its free end, provision is made for a light emission surface, which is located at right angles to the extension of the retaining lug. Provided that the retaining hook encompasses the typical 90° deflection, which is the norm, the beveled surface located on the outside of the curvature, can be at an angle of 45° to the two axes of the two legs of the hook element. As is known per se, the angle of deflection or curvature can also be considerably larger or smaller than 90°. Typically, retaining hooks including an angle of deflection or curvature a of 60°≦α≦120° are used.

The hook element itself advantageously encompasses a flat-oval cross section, a flat-rectangular cross section including rounded edges or a concave cross section, which runs in the longitudinal direction thereof, thus the shape of an elongate, angled spoon. Together with the selection of the suitable PMMA or polycarbonate material, a good bending stiffness or a tension, respectively, can thus be reached in the area of the tissue retaining lug of 100 to up several hundred N.

The polycarbonate material, which has already been tested, or the polymethylmethacrylate material, which has been tested, respectively, including very smooth outer surfaces on the hook element provide for a good light distribution from the emission surface of the light source, in one embodiment of the white light LED, to the light emission surface on the tissue retaining lug. In spite of the comparatively very low output of the light source, a good in situ illumination of the surgical site can be ensured with this. The handle, to which the hook element is flange-mounted or attached, respectively, for example via a form-fit coupling, the bayonet lock mentioned above, a screw closure, a press lock or also a ring snap lock, is designed as hollow body, which accommodates an energy storage means, in one embodiment a battery or an accumulator. The power supply circuit board including a supply circuit and light source is inserted between the end, which faces the hook element, and the energy storage means in the interior. In the case of tested models of the subject matter of the invention, the handle of the hand piece is provided on the bottom side with a removable cover, for example a cover including threaded or snap lock, which operates a switch for turning the light source on/off at the same time by further twisting.

In the case of one tested alternative embodiment of the invention, the connection section of the hook element to the handle is embodied as a cap, which can be attached to the light emission side of the handle in a form-fit manner, and to the closed front surface of which, the cap bottom, a switch guide plate is molded, by which a control button for turning on/turning off the light source, which projects forward on the upper front surface of the handle housing and which is spring pre-loaded, can be actuated in response to the twisting of the retaining hook relative to the housing of the handle.

To ensure a concentric distribution of light, which allows in one embodiment to get by with white light LEDs having a comparatively low output in response to a very good spot light illumination of the surgical site, provision is made in one embodiment of the invention on the outer side of the hook, which faces away from the curvature of the hook element on the end side, running in longitudinal direction thereof, for a light distribution line, including a narrowing free end, which tapers, at which the spot light escapes. This light distribution line can encompass the shape of a stylus, which is molded in one piece, including a round cross section or a cross section, which is rounded at least on the outer side. In this case, the hook element including the molded light distribution line can be produced in a highly-polished injection die from the mentioned plastic material in a single operating process. The LED white light source should supply a luminous flux in the range of 601 m≦φᵥ≦2001 m in response to a current consumption of 100 mA≦I_{F}≦1 A, which is suitable for battery/accumulator operation. For example, suitable LEDs are the types OSLON SSL, LCW CQ7P and LUW CQ7P, which are currently offered by OSRAM Opto Semiconductors.

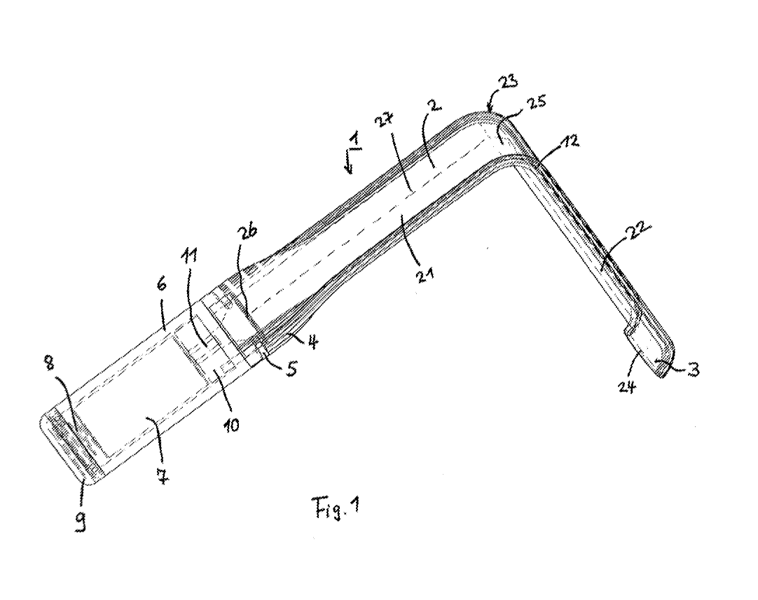

With reference to its retaining function, a medical retaining hook 1 according to FIG. 1 consists of a one-piece, two-legged hook element 2, the leg 21 on the handle side and the free leg 22 of which are connected in one piece via a curvature 23 of 90° in the illustrated example or of substantially 90°. The angle of curvature can also be larger or smaller than 90°, can thus be chosen for instance in the range of 60°≦α≦120°. In the illustrated embodiment alternative, the hook element 2 encompasses a concave curvature 12, which is directed inwardly or downwardly, respectively, thus the shape of an elongate spoon, wherein it goes without saying that the free edges are rounded. On its lower free end, the hook element 2 encompasses a tissue retaining lug 3, which is bent inwardly in the direction of the handle and the free end surface 24 of which forms the light emission surface. The cross section of the hook element 2 is not limited to the illustrated form of an elongate spoon. The cross sectional form can also be long-oval or rectangular including rounded edges. In the area of the curvature 23, a beveled surface 25, which is at an angle of 45° to the respective directions of extension of the two legs 21, 22 in response to a 90° bend of the hook element 2, and which additionally ensures a good distribution of light, can be present on the outside, in one embodiment when an oval cross section is chosen.

In the area of the handle 6, the hook element 2 is provided with an expansion 4, which ends in a substantially circular cross section, the outer diameter of which is adapted to the diameter of the handle 6. The handle 6 is designed, for example, as a metallic sleeve, for example of stainless steel, on the end of which, which faces the hook element 2, the supply circuit board 10 including a supply circuit for a white light emitting LED is inserted as light source 11. A form-fit coupling 5, for example in the form of a bayonet closure, connects the hook element 2 to the handle 6. The light source 11 can be surrounded by a ceramic frame or the corresponding supply circuit board 10, respectively, can be made of ceramic, so that a sufficient heat distribution or removal, respectively, is ensured, in one embodiment when the LED frame or the circuit board, respectively, is also provided with an aluminum collar. The sleeve-like handle 6 serves to accommodate an electrical energy storage device 7, for example a common battery or an accumulator. On the bottom side, thus in the area of the free end of the handle 6, a pressure spring 8, which presses the electrical energy storage device 7 against the corresponding terminal of the circuit board 10 or of the supply circuit, respectively, for the LED 11 is inserted on the inside. On the free end, the sleeve of the handle 6 is closed by using a cover 9 or clamping cover, which can be screwed thereto, into which a switch element (not illustrated) is integrated so that the LED 11 can be activated in response to the twisting or further turning, respectively, of the cover 9 in locking direction.

The white light emitted by the light source 11 is irradiated into the end surface 26 of the hook element 2 on the connection side and is guided within the plastic material of the hook element 2; it then escapes again on the light emission surface 24 of the tissue retaining lug 3 and thus ensures a good illumination of the surgical site. To ensure a distribution of the light, which is emitted by the light source 11, with as little loss as possible, in one embodiment it is advantageous to move the light entry surface 26 on the hook element as closely to the emission surface of the LED as possible. It can also be advantageous to provide for an integrated lens (not illustrated) either on the light emission surface of the LED or on the light entry surface 26 of the hook element 2.

An alternative embodiment consists in introducing an optical fiber 27 (illustrated by using dashed lines) as being integrated into the material of the hook element 2, so as to nonetheless ensure an optimal illumination of the surgical site in response to a reduction of the output of the light source, which is then possible.

In the case of one exemplary embodiment of the invention according to FIG. 2 with partial FIGS. 2A to 2D, the hook element 2 consists substantially of a cap 30, which is attached to the handle 6 so as to be adapted accordingly in a form-fit manner, wherein a slight inner cone of for example 2° (see FIG. 2D) facilitates the form-fitted sliding onto the end of the handle on the light side and thereby ensures a good, largely clearance-free fit of the cap 30 on the handle. To ensure a positionally accurate orientation of the light source 11 onto the hook element 2, which will be described below, including an integrated additional light conductor, a for example dovetail-shaped attachment is embodied on the front surface of the handle 6 on the light side, to which a corresponding recess 33 in the bottom of the cap 30 corresponds, so that the cap can only be attached to the handle 6 in a desired rotation angle position and can then subsequently be twisted only in an angular range of 90° for reasons, which will be explained below, due to a radial strop 34.

The free leg 22 of the hook element 2, which—similarly as in FIG. 1—has the form of an elongate spoon including a retaining lug 3, which is angled in the direction of the handle, and which projects at an angle of 75°≦α≦105°, at an angle of 90° to the cap 30, is attached to the outer jacket of the cap 30.

To ensure a concentrated distribution of light, which is further improved as compared to FIG. 1, a depression or groove 35, which runs centrally in longitudinal extension of the leg 22 and which leads from the light source 11 to the free tip 36, the light emission point, for holding and guiding a light guide 19, is molded on the spoon bottom of the leg 22, wherein the front free end of the light distribution line 19 is embodied as a conically narrowed tip 28. It can be seen well from FIGS. 2A, 2B and 2C that the light emission takes place via a very slight, virtually punctiform light decoupling surface 36. To reach an even sharper focusing of the light, which is irradiated by the light source 11 into the leg 22 of the retaining hook 2, the stylus-like light guide 19 can be provided on the end side of its punctiform light decoupling surface 36 with a lens 40, which is suggested in FIG. 2B, in one embodiment a convex lens, via which the light decoupling takes place. The light is then virtually emitted as punctiform light beam, so that a desired pinpoint illumination of the surgical site is realized optimally.

The cap 30, together with the leg 22, forms the retaining hook, which, including the light guide 19, which is guided in the groove 35, is provided in one piece in a highly polished injection die of the mentioned highly light-conducting polycarbonate or polymethylmethacrylate material having a high optical clarity and mechanical load-bearing capacity.

As explained above, the retaining hook, substantially consisting of the cap 30 and the leg 22 including light guide 19, can be twisted or pivoted, respectively, in a certain angle, for example of 90°, against the handle 6. By using this limited relative rotary motion between the retaining hook and the handle, a turn on/turn off for the light source 11 or for the power supply circuit of the light source 11, respectively, can be actuated. For this purpose, provision can be made on the front surface of the handle, which faces the hook element 2, for a spring pre-loaded push-button switch (not illustrated) including a rounded head, which is guided so as to engage with a link groove or funnel-like bevel 32, which is recessed on the inner bottom of the cap 30. As soon as the hook element 2 has reached the correct orientation position between light source and the irradiation end of the light guide 19 in response to the twisting, the spring pre-loaded head jumps into a hole-like depression, a tapped blind hole in the bottom area of the cap 30 on the inner side, whereby the light source 11 turns on. With this characteristic of the design, exposed sections, connection grooves and the like are no longer required, in one embodiment in the area of the handle 6, which is particularly advantageous for the sterilization and sterility of the handle. The hook element 2 itself, with cap 30 and leg 22, including light guide 19, is provided as pre-sterilized disposable item.

In the daily routine operation of a hospital, the operating and cost effort for the device sterilization in the surgical area, among others, is a considerable cost factor. The invention makes a contribution to the cost reduction herein. In the case of a surgical intervention in body cavities (abdomen, thorax), in plastic surgery or also in dentistry, a plurality of retaining hooks of the type in question herein, of course in different sizes, are routine required for every surgical appointment, which currently need to be taken apart, cleaned and sterilized subsequently. The invention does not only provide the advantage of a medical retaining hook, which can be used without any cables and without any problems, but also the advantageous possibility to use the hook element, which can be attached to the handle including an energy storage means or device—as mentioned—as presterilized disposable item, which must neither be cleaned nor does it not need to be sterilized after the surgical intervention. In the case of one embodiment of the invention, which is presently in the testing phase, the handle is still made as a sleeve-like element made of stainless steel. However, it is also possible to produce the handle as a plastic injection molding part, so that a complete disposal of the entire hook element 2 after a surgical intervention becomes possible. A one-piece production of the retaining hook, including the handle is also possible, wherein the equipping with the LED light source 11, including supply circuit board and electrical energy source can be done from the underside, that is, from the side of the cover 9. In this case, an electrical energy source with a small output is also possible, which would have to be disposed of separately as a single element after being used.

It is to be understood that the features of the various exemplary embodiments described herein may be combined with each other, unless specifically noted otherwise.

Although specific embodiments have been illustrated and described herein, it will be appreciated by those of ordinary skill in the art that a variety of alternate and/or equivalent implementations may be substituted for the specific embodiments shown and described without departing from the scope of the present invention. This application is intended to cover any adaptations or variations of the specific embodiments discussed herein. Therefore, it is intended that this invention be limited only by the claims and the equivalents thereof.

1-19. (canceled)

20. A medical device including a medical retaining hook for holding tissue open during surgical interventions, comprising:
a hook element removably attached to a handle having a light source, the hook element constructed as a light conductor that guides light emitted from the light source along the hook element, the hook element made of a light-conducting polycarbonate or polymethylmethacrylate.

21. The device of claim 20, the handle having a hollow body, including an electrical storage device including the light source coupled to an energy source and supply circuit, where light emitted from the light source is irradiated onto an irradiation surface on a first end of the light conductor and transmitted to a second end of the light conductor.

22. The device of claim 21, comprising a form-fit coupling that couples the hook element to the handle.

23. The device of claim 20, wherein the light source is a light emitting diode.

24. The device of claim 23, wherein the light-emitting diode (LED) is a white light emitting LED.

25. The device of claim 21, comprising a lens inserted between the light source and the irradiation surface of the hook element.

26. The device of claim 20, the hook element having a curvature of 70°≦α≦110°, and a flat-oval cross section, as well as a tissue retaining lug bent inwardly on the free end, in the direction of the handle.

27. The device of claim 20, the hook element having a curvature of 70°≦α≦110°, and a flat-rectangular cross section comprising rounded edges as well as a tissue retaining lug bent inwardly, on the end, in the direction of the handle.

28. The device of claim 27, wherein longitudinal edges of the hook element are bent in the same direction.

29. The device of claim 20, wherein the hook element encompasses a curvature of 70°≦α≦110°, a concave cross section running in the longitudinal direction thereof in the form of an elongate angled spoon, and a tissue retaining lug bent inwardly, on an end, in the direction of where the light source is attached.

30. The device of claim 20, comprising an optical fiber, positioned along the hook element and which defines the light conductor, and is integrated into the material of the hook element.

31. The device of claim 30, comprising a lens operably positioned on the first end of the the hook element in the area of light emission from the optical fiber.

32. The device of claim 20, the handle comprising a cover including an integrated switch for turning on/turning off the light source.

33. The device of claim 20, comprising a light distribution line, that defines a light guide from the light source into an end of the hook element, attached along an outer surface of the hook element.

34. The device of claim 33, wherein the light distribution line encompasses a curved cross section and is embodied so as to taper conically comprising a narrowed light decoupling surface that provides a dot-like light emission, in the area of its free light emission end.

35. The device of claim 34, wherein the light distribution line encompasses a round cross section.

36. The device of claim 34, wherein the light distribution line is made of the same light-conducting material and is made so as to be connected in one piece with the hook element.

37. The device of claim 33, wherein the hook element is embodied in one piece with a retaining cap, the inner diameter of which is adapted to the end area of the handle on the light emission side.

38. The device of claim 37, wherein, on the inner side of the bottom, the retaining cap encompasses a connecting member track, which runs so as to rise/drop in orientation of the handle, or which encompasses a conical depression, with which a spring pre-loaded push button switch on the front surface, which is covered by the holding cap, engages for tuning on/turning off the light source in response to a relative twisting of hook element and handle when the hook element is attached to the handle.

39. The device of claim 34, comprising a lens arranged on the narrowed light decoupling surface.

40. The device comprising:
a handle having a hollow body, having an electrical storage device including a light source coupled to an energy source and supply circuit, where light emitted from the light source is irradiated onto an irradiation surface on a front end of the light conductor and transmitted to a second end of the light conductor;
a hook element attached to the handle, constructed as a light conductor that guides light emitted from the light source along the hook element, the hook element made of a light-conducting polycarbonate or polymethylmethacrylate;
wherein the hook element encompasses a curvature of 70°≦α≦110°, a concave cross section running in the longitudinal direction thereof in the form of an elongate angled spoon, and a tissue retaining lug bent inwardly, on the free second end, in the direction of where the light source is attached; and
a lens operably positioned at an end of the the hook element.
